(19) United States
(12) Patent Application Publication
(10) Pub. No.: US 2023/0113554 A1
(43) Pub. Date: Apr. 13, 2023

(54) NON-STICK ANTIBIOTIC GELS

(71) Applicant: AO TECHNOLOGY AG, Chur (CH)

(72) Inventor: Matteo D'ESTE, Davos Platz (CH)

(73) Assignee: AO TECHNOLOGY AG, Chur (CH)

(21) Appl. No.: 17/802,992

(22) PCT Filed: Dec. 10, 2020
(86) PCT No.: PCT/EP2020/085466

§ 371 Date: Aug. 29, 2022

(30) Foreign Application Priority Data
Dec. 16, 2019 (CH) 01628/19

(51) Int. Cl. A61K 9/107; A61K 9/06; A61K 47/44; A61K 47/24; A61K 47/38; A61K 47/10; A61K 31/7036; A61K 38/14; A61K 38/12; A61K 47/12; B01F 23/41; B01F 23/411; B01F 31/80

(52) U.S. Cl. CPC A61K 9/107; A61K 9/06; A61K 47/44; A61K 47/24; A61K 47/38; A61K 47/10; A61K 31/7036; A61K 38/14; A61K 38/12; A61K 47/12; B01F 23/4105; B01F 23/4111; B01F 23/4145; B01F 31/89; B01F 2101/22

(57) ABSTRACT

A method of producing a pharmaceutical gel emulsion, wherein the emulsion is an oil-in-water gel emulsion, comprising the steps of forming an oil-in-water emulsion comprising at least one pharmaceutically acceptable oil, at least one aqueous phase, at least one osmotic agent, at least one emulsifying agent, mixing a gelling polysaccharide with the oil-in-water emulsion and allowing the resulting mixture to form the pharmaceutical gel emulsion, optionally mixing an bioactive agent into the pharmaceutical gel emulsion.

### TECHNICAL FIELD

The present invention relates to gel emulsions that are applied to bone defects and which can be handled easily.

### PRIOR ART

FR 3062 059 discloses a base cosmetic formulation emulsion comprising an aqueous phase of 15 to 50 wt % of glycol, 0 to 50 wt % of water and 0.2 to 5 wt % of a gelling agent and an oily phase of 15 to 60 wt % of emollient such as oil and 0.2 to 10 wt % of an emulsifying agent.

CN 104436286 discloses a hemostatic textile (gauze) made by depositing a spinning dope into a coagulation bath, where the spinning dope comprises a gelling polysaccharide such as sodium alginate, CMC or pectin, an emulsifying agent such as polysorbate or poloxamer, an oil such as soybean oil, a polyol such as xylitol.

When undergoing surgical interventions, especially in the field of treating skeletal trauma and joint replacement surgery, there exists the necessity of preventing or eliminating infection at the site of intervention where a bone defect is present.

A general solution to this problem is applying a bioactive or antibiotic composition at the site of intervention to mitigate the risk of infection by means of increasing the local antibiotic concentration and at the same time keeping a low systemic concentration, thereby limiting the off-target effects. Likewise, the same approach can be employed for infection eradication. In both infection prevention, eradication, and prevention of re-infection the local antibiotics can be conveniently combined with systemic antibiotic administration. Ideally, such a composition should be able to be effectively delivered to the site of intervention and remain there for a certain duration, after which it should disintegrate or be absorbed by the surrounding tissues, thus preventing exacerbation of inflammation and fibrous encapsulation or scar tissue formation.

Such compositions may be in the form of pastes, cremes, gels or ointments which are delivered in a first step via a syringe or a spatula into, or the vicinity of, the site of intervention, and which then are molded into their final shape by hand.

Surgeons wear gloves during the surgical interventions and a known issue with existing compositions is that of the compositions strongly sticking to the outer glove surface, especially when already covered in blood, which on one hand complicates a complex procedure that must be carried out under time constraints and on the other hand leads to the loss of composition sticking to the gloves which would otherwise have been available for application at the site of intervention.

Additionally, such compositions must have certain rheological properties which allow the compositions to be applied effectively while at the same time staying in place after being applied.

### SUMMARY OF THE INVENTION

The present invention provides for an emulsion gel, in particular an oil-in-water emulsion gel that exhibits excellent rheological, in particular viscoelastic, properties that allow the emulsion gel to be applied effectively, stay in place after application and which exhibits low stickiness, i.e. adhesion, to plastic surfaces such as medical gloves. The emulsion gels according to the present invention are cohesive and do not fall apart after contact with body fluid (FIG. 4), and are therefore suitable to fill bone defects and to be spread on implantable devices including metal implants, orthopedic devices, artificial joints, pace makers, and in all situations where biomaterials are implanted in the body of an animal or human. The present invention further provides a method of producing such emulsion gels, as well as the use thereof as a medicament.

It is an object of the present invention to provide a method of producing a pharmaceutical gel emulsion, wherein the emulsion is an oil-in-water gel emulsion, comprising the steps of forming an oil-in-water emulsion comprising at least one pharmaceutically acceptable oil, at least one aqueous phase, at least one osmotic agent, at least one emulsifying agent, mixing a gelling polysaccharide with the oil-in-water emulsion and allowing the resulting mixture to form the pharmaceutical gel emulsion, optionally mixing an bioactive agent into the pharmaceutical gel emulsion.

In an embodiment of the method of producing a pharmaceutical gel emulsion, wherein the emulsion is an oil-in-water gel emulsion, the method comprises the steps of

a. forming a first dispersion by dispersing at least one emulsifying agent in an aqueous phase,

b. forming an oil-in-water emulsion by dispersing at least one pharmaceutically acceptable oil and at least one osmotic agent in the first dispersion,

c. mixing a gelling polysaccharide with the oil-in-water emulsion and allowing the resulting mixture to form the pharmaceutical gel emulsion,

d. optionally mixing an bioactive agent into the pharmaceutical gel emulsion.

In an embodiment of the method of producing a pharmaceutical gel emulsion, wherein the emulsion is an oil-in-water gel emulsion, the method comprises the steps of

a. forming a first dispersion by dispersing at least one emulsifying agent in at least one pharmaceutically acceptable oil,

b. forming an oil-in-water emulsion by dispersing at least one aqueous phase and at least one osmotic agent in the first dispersion,

c. mixing a gelling polysaccharide with the oil-in-water emulsion and allowing the resulting mixture to form the pharmaceutical gel emulsion,

d. optionally mixing a bioactive agent into the pharmaceutical gel emulsion.

In the context of the present invention, the term “gel” refers to a material having either 1. a shear storage modulus (G′) above its shear loss modulus (G″), i.e. G′>G″, when measured with a rheometer with a strain sweep at amplitude within the linear viscoelastic range, or 2. a storage modulus above 1000 Pa independently of its loss modulus, when measured with a rheometer with a strain sweep at amplitude within the linear viscoelastic range.

In the context of the present invention, the term “emulsion” refers to a mixture of at least two immiscible liquids, where at least one liquid is dispersed in at least one other liquid. The dispersed liquid forming the non-continuous phase is generally referred as to as the dispersed phase and the liquid forming the phase in which the dispersed phase is dispersed is generally referred to as the continuous phase.

In a preferred embodiment of the method of producing a pharmaceutical gel emulsion according to the present invention, the pharmaceutical gel emulsion is an oil-in-water gel emulsion, i.e. an oily phase is dispersed in an aqueous continuous phase.

The first dispersion is formed by dispersing at least one emulsifying agent in an aqueous phase or in at least one pharmaceutically acceptable oil which may be achieved for example by combining the at least one emulsifying agent and an aqueous phase or an at least one pharmaceutically acceptable oil and subjecting the thus resulting mixture to shear. The shear necessary for the formation of a dispersion of the at least one emulsifying agent in the aqueous phase or the at least one pharmaceutically acceptable oil may be provided by for example a sonication device or a microfluidizer. Alternatively, the shear may be provided by microfiltration, i.e. by forcing the resulting mixture of the at least one emulsifying agent and an aqueous phase across a filter having a pore size of less than 1000 nm, of less than 200 nm.

In the case the dispersion of the at least one emulsifying agent is obtained with a sonication device, said sonication must be performed until a homogeneous dispersion is obtained and no residual solid or particles are present. To achieve this homogeneous distribution, for a batch size for 50 ml circa the sonication device may be set at power between 0.1 and 120 W, or between 40 and 70 W, and preferably of about 60 W, and/or a frequency between 15 and 40 kHz, preferably between 19 and 21 kHz and/or an amplitude (=intensity) between 25 and 250 μm for a duration of 2 up to 7 minutes, until homogeneity.

In the case the dispersion of the at least one emulsifying agent is obtained with a by microfiltration, a filter having a pore size of between 10 μm and 50 nm, preferably of between 1 μm and 200 nm may be used.

The at least one emulsifying agent may be chosen from lecithin, which may be vegetal or animal, surfactants, and other emulsifying agents. Suitable emulsifying agents may be selected from the list comprising, but not limited to: egg lecithin; soybean lecithin; non-GMO lecithin; natural phospholipids, synthetic phospholipids, sphingomyelin, natural extracts containing sphingomyelin, phosphatidyl glycerol and natural extracts containing it, phosphatidylethanolamine and natural extracts containing it, 1,2-dimyristoyl-sn-glycero phosphocholine, 1,2-dipalmitoyl-sn-glycero-3-phosphocholine, 1,2-distearoyl-sn-glycero phosphocholine, 1,2-dioleoyl-sn-glycero-3-phosphocholine, 1-palmitoyl-2-oleoyl-sn-glycero-3-phosphocholine, 1,2-dierucoyl-sn-glycero-3-phosphocholine, 1,2-dimyristoyl-sn-glycero-3-phospho-rac-glycerol or its sodium salt, 1,2-dipalmitoyl-sn-glycero-3-phospho-rac-glycerol or its sodium salt, 1,2-distearoyl-sn-glycero-3-phospho-rac-glycerol or its sodium salt, 1,2-dioleolyl-sn-glycero-3-phospho-rac-glycerol or its sodium salt, hydrogenated phospholipids, rapeseed lecithin; sunflower lecithin; lysolecithin; phosphatidylcholine, natural extracts containing phosphatidylcholine, sorbitan monoesters (also known as Span), polyethoxylated monoesters (also known as Tween), polysorbates, fatty acids and fatty acids salts, palmitic acid, oleic acid, phosphocholines, sn-glycero-3-phosphocholine.

Further emulsifying agents known to the person skilled in the art and suitable for use in the present invention include, without limitation, gum Arabic, agar, alginates, Acacia, Carbomer Copolymer, Carbomer Interpolymer, Cholesterol, Coconut Oil, Diethylene Glycol Stearates, Ethylene Glycol, Stearates, Glyceryl Distearate, Glyceryl Monolinoleate, Glyceryl Monooleate, Glyceryl Monostearate, Lanolin Alcohols, Lecithin, Mono- and Diglycerides, Poloxamer, Polyoxyethylene 50 Stearate, Polyoxyl 10 Oleyl Ether, Polyoxyl 20 Cetostearyl Ether, Polyoxyl 35 Castor Oil, Polyoxyl 40 Hydrogenated Castor Oil, Polyoxyl 40 Stearate, Polyoxyl Lauryl Ether, Polyoxyl Stearyl Ether, Polysorbate 20, Polysorbate 40, Polysorbate 60, Polysorbate 80, Propylene Glycol Monostearate, Sodium Cetostearyl Sulfate, Sodium Lauryl Sulfate, Sodium Stearate, Sorbitan Monolaurate, Sorbitan Monooleate, Sorbitan Monopalmitate, Sorbitan Monostearate, Sorbitan Sesquioleate, Sorbitan Trioleate, Stearic Acid, and Wax Emulsifying.

The above emulsifying agents may be used alone or in combination, like for example egg lecithin/sodium oleate 40/1, egg lecithin containing 80 weight % phosphatidyl choline in combination with natural extract of sodium oleate containing 50 weight % or above sodium oleate, egg lecithin containing 70 weight % phosphatidyl choline in combination with sodium oleate from synthesis or from extraction, egg lecithin containing 96 weight % phosphatidyl choline in combination with sodium oleate from synthesis or from extraction, egg lecithin containing 98 weight % phosphatidyl choline in combination with sodium oleate from synthesis or from extraction.

The aqueous phase suitable for preparing the gel emulsion of the present invention may be any of the commonly used aqueous solvents well known by those of ordinary skill in the art such as water or aqueous solutions of salts or buffers. Preferably, the aqueous phase may be selected from the group consisting of water, pure or ultrapure water (including water for injections), aqueous buffer solutions, acid solutions, basic solutions, salt solutions, saline solution, and glucose salt solution. Aqueous buffer solutions having a pH of from 4.0 to 9.5 are for example sodium acetate buffer, phosphate buffer saline, Tris buffer, sodium phosphate buffer, MOPS, PIPES, MES and potassium phosphate (e.g. in the range of 25 mM to 500 mM and in the pH range of 4.0 to 9.5). The aqueous solvent or aqueous buffer solution can also contain up to 60% of organic solvents. Examples of organic solvents include, but are not limited to, alcohols, DMF, DMSO, NMP, Acetonitrile, Ethanol, Methanol, Propanol (n- or iso-), butanol, dioxane, THF. Preferably the aqueous phase or aqueous buffer solution are free of organic solvents, meaning that the organic solvent content of the aqueous solvent or aqueous buffer solution does not exceed 0.1% by weight, based on the total weight of the aqueous solvent or aqueous buffer solution.

The oil-in-water emulsion is formed by dispersing at least one pharmaceutically acceptable oil or at least one aqueous phase and at least one osmotic agent such as for example a polyol in the first dispersion, which may be achieved for example by combining the first dispersion with the at least one pharmaceutically acceptable oil or at least one aqueous phase and the at least one osmotic agent, in any order or simultaneously, and subjecting the thus resulting mixture to shear. The shear necessary for the formation of an oil-in-water emulsion may be provided by for example a sonication device or a microfluidizer.

Alternatively, the shear may be provided by microfiltration, i.e. by forcing the resulting mixture of the at least one emulsifying agent, at least one aqueous phase, at least one pharmaceutically acceptable oil and the at least one osmotic agent across a filter having a pore size of 10 μm or less, of 1000 nm or less, or of 200 nm or less.

In the case the oil-in-water emulsion is obtained with a sonication device, the sonication device, said sonication must be performed until a homogeneous emulsion is obtained and no separation of water and oil phase is visible. To achieve this homogeneous distribution, for a batch size for 50 ml circa the sonication device may be set at power between 0.1 and 120 W, or between 40 and 70 W, preferably of about 60 W, and/or a frequency between 15 and 40 kHz, preferably between 19 and 21 kHz and/or amplitude (=intensity) between 25 μm and 250 μm, for a duration of 2 up to 7 minutes, until homogeneity.

In the case the oil-in-water emulsion is obtained by microfiltration, a filter having a pore size of between 10 μm and 50 nm, preferably of between 1 μm and 200 nm may be used.

The at least one pharmaceutically acceptable oil may be chosen from one or more oil, for example suitable mineral oil or animal or plant oils comprising triglycerides or diglycerides. Suitable oils that may be used as pharmaceutically acceptable oil include, without limitation, Omega-3-Acid Triglycerides, lipoid medium chain triglycerides, ethyl oleate, isopropyl myristate, isopropyl palmitate, light mineral oil, mineral oil, myristyl alcohol, purified fish oil, jojoba oil, avocado oil, almond oil, olive oil, purified olive oil, sesame oil, peanut oil, cottonseed oil, wheat germ oil, rapeseed oil, canola oil, sunflower oil, safflower oil, soybean oil, corn (maize) oil, cottonseed oil, rice bran oil, camellia oil, castor oil, grape seed oil, green tea seed oil, macadamia nut oil, palm oil, and rosehip oil, as well as any other suitable pharmaceutically acceptable oil know to the person of ordinary skill in the art.

Osmotic agents and/or tonicity agents, referred to collectively as osmotic agents for the purposes of this application, are well known to a person skilled in the art. Osmotic agents/tonicity agents include for example, without limitation, dextrose, glycerin, hydroxpropyl betadex, mannitol, potassium chloride, sodium chloride, and polyols. In a preferred embodiment, polyols are chosen as osmotic/tonicity agents.

More preferably, the at least one polyol may be chosen from low molecular weight polyols such as glycerol, propylene glycol, erythritol 1,2,4-butanetriol or oligo ethylene glycol. Exemplary polyether polyols are polyethylene glycol (PEG) and polypropylene glycol (PPG).

The pharmaceutical gel emulsion is formed by mixing a gelling polysaccharide with the oil-in-water emulsion and allowing the resulting mixture to form the pharmaceutical gel emulsion, which may be achieved for example by combining the gelling polysaccharide with the oil-in-water emulsion, stirring to mix the gelling polysaccharide with the oil-in-water emulsion and allowing the resulting mixture to rest so that the gelling polysaccharide may gel in the presence of the water comprised in the oil-in-water emulsion.

In the case where the gelling polysaccharide is a cellulose derivative such as for example carboxymethylcellulose (CMC), the gelling polysaccharide is added to the oil-in-water emulsion, stirred to mix the gelling polysaccharide with the oil-in-water emulsion and the resulting mixture is allowed to rest for at least one hour so that the gelling polysaccharide may gel in the presence of the water comprised in the oil-in-water emulsion, and where preferably the sequence of stirring and subsequent resting is repeated at least once, twice, thrice or even more times, after which the resulting mixture is further preferably left to rest for another 12 hours or 24 hours.

In a preferred embodiment of the method of producing a pharmaceutical gel emulsion according to the present invention, the pharmaceutical gel emulsion obtained in step c. may further be sterilized, such as for example by heat sterilizing such as steam sterilizing the pharmaceutical gel emulsion.

In a preferred embodiment of the method of producing a pharmaceutical gel emulsion according to the present invention, the pharmaceutical gel emulsion includes a bioactive agent and is formed by mixing a bioactive agent into the pharmaceutical gel emulsion.

In the case where a bioactive agent is added and the pharmaceutical gel emulsion is sterilized, the sterilizing step is preferably carried out before the inclusion of the bioactive agent in order to avoid thermal inactivation of the bioactive agent. It is understood that in this case, the bioactive agent is added under sterile conditions to the sterilized pharmaceutical gel emulsion.

The one or more bioactive agent may be selected from inorganic salts to a macromolecular compound or a small molecule compound, such as strontium salts, strontium ranelates, bisphosphonates, etidronates, clodronates tiludronates, pamidronates, neridronates, olpadronates, alendronates, ibandronates, risedronates zoledronates, icaritin and analogues, kartogenin and analogues.

Suitable bioactive agents for use in the method of producing a pharmaceutical gel emulsion according to the present invention may be selected from compounds such as growth factors, enzymes, antitumoral drugs, anti-inflammatory drugs, antiviral drugs, antifungal drugs, anesthetics, anti-neoplastic drugs, antimitotic drugs, analgesics, narcotics, antithrombotic drugs, anticoagulants, haemostatic drugs, peptides, proteins, oligo- and poly-nucleotides, antibiotics, antibacterials, antimicrobics, disinfectants, antiseptics, bactericidal and bacteriostatic substances for infection prevention and infection treatment such as aminoglycosides in particular gentamicin, ansamycins, carbacephem, carbapenems, cephalosporins (particularly cephalosporin of first, second, third generation), glycopeptides, glycylcyclines, lipiarmycins (such as fidaxomicin), lincosamides, lipopeptide, macrolides, monobactams, nitrofurans, oxazolidonones (such as linezolid), penicillins, penicillin combinations, polymixins, polypeptides, rifamycins, quinolones, sulfonamides, tetracyclines, drugs against mycobacteria. Specific examples of such substances include: amikacin, gentamicin, gentamicin sulfate, silver, colloidal silver, silver powder, silver nanoparticles, silver compounds, silver releasing compounds, kanamycin, neomycin, netilmicin, tobramycin, paromomycin, spectinomycin, geldanamycin, herbimycin, ‘rifaximin’, streptomycin, loracarbef, ertapenem, doripenem, ‘imipenem’/cilastatin, meropenem, cefadroxil, cefazolin, ‘cefalotin’ or cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, amoxicillin/clavulanate, ampicillin/sulbactam, piperacillin/tazobactam, ticarcillin/clavulanate, bacitracin, colistin, polymyxin B, ciprofloxacin, enoxacin, gatifloxacin, levofloxacin, lomefloxacin, moxifloxacin, nalidixic acid, norfloxacin, ofloxacin, trovafioxacin, grepafloxacin, sparfloxacin, temafloxacin, mafenide, sulfacetamide, sulfadiazine, silver sulfadiazine, sulfadimethoxine, sulfamethizole, sulfamethoxazole, ‘sulfanilimide’ (archaic), sulfasalazine, sulfisoxazole ‘trimethoprim’-sulfamethoxazole (co-trimoxazole) (TMP-SMX), sulfonamidochrysoidine (archaic), demeclocycline, doxycycline, minocycline, oxytetracycline, tetracycline, clofazimine, dapsone, capreomycin, cycloserine, ethambutol, ethionamide, isoniazid, pyrazinamide, ‘rifampicin’ (rifampin in US), rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, trimethoprim, Dalbavancin, iclaprim, cethromycin, oritavancin, or ramoplanin.

Additional bioactive agents for use in the method of producing a pharmaceutical gel emulsion according to the present invention may be selected from entities such as microorganisms, viruses, phages, bacteriophages, bactericidal microorganisms.

Suitable bioactive agents for use in the method of producing a pharmaceutical gel emulsion according to the present invention may further be selected from the group consisting of (a) compounds such as proteins or (poly) peptides, such as endolysins, enzybiotics, lysostaphin, lysosome, Endo-β-N-acetylglucosaminidase (Endoglycosidase H, EC 3.2.1.96), N-acetylmuramidase (lysozyme-like, EC 3.2.1.17), Endopeptidase, N-acetylmuramoyl-L-alanine amidase (T7-like, EC 3.5.1.28), γ-D-glutaminyl-L-lysine endopeptidase (EC 3.4.14.13) transglycosylases, amidases, such as bacteriophage T7 gp3.5, endopeptidases, lysins extracted from natural sources, lysins produced by genetic engineering, erythropoietin (EPO), interferon-alpha, interferon-beta, interferon-gamma, growth hormone (human, pig, cow, etc.), growth factors such as transforming growth factor-beta (TGF-beta), fibroblast growth factor (bFGF), vascular endothelial growth factor (VEGF), and the like, bone morphogenetic proteins (BMPs), BMP2, BMP7, OP1, dexamethasone and analogues, corticosteroids, fibronectin, fibrinogen, thrombin, proteins, GDFS, SDF1, CCL5, homing factors, TGS6, growth hormone releasing factor, nerve growth factor (NGF), granulocyte-colony stimulating factor (G-CSF), granulocyte macrophage-colony stimulating factor (GM-CSF), macrophage-colony stimulating factor (M-CSF), blood clotting factor, insulin, oxytocin, vasopressin, adrenocorticotropic hormone, epidermal growth factor, platelet-derived growth factor (PDGF), prolactin, luliberin, luteinizing hormone releasing hormone (LHRH), LHRH agonists, LHRH antagonists, somatostatin, glucagon, interleukin-1 (IL-1), interleukin-1 receptor antagonist (IL-1RA), interleukin-2 (IL-2), interleukin-11 (IL-11), gastrin, tetragastrin, pentagastrin, urogastrone, secretin, calcitonin, enkephalins, endorphins, angiotensins, thyrotropin releasing hormone (TRH), tumor necrosis factor (TNF), tumor necrosis factor related apoptosis inducing ligand (TRAIL), heparinase, human atrial natriuretic peptide (hANP), glucagon-like peptide (GLP-I), renin, bradykinin, bacitracins, polymyxins, colistins, tyrocidine, gramicidins, cyclosporins and synthetic analogs thereof, antibodies, monoclonal antibodies, polyclonal antibodies, and cytokines; and (b) vaccines; and (c) nucleic acid such as small interference RNA (siRNA), micro RNA (miRNA), extracts from extracellular vesicles, extracts from exosomes, extracts from microvesicles, plasmid DNA, and antisense oligodeoxynucleotide (AS-ODN), viral vectors including adenoviruses, adenoassociated viruses, integrating viral vectors such as for example retroviruses, lentiviruses, non-viral vectors such as liposomes, charged polymers, inorganic salts capable of binding to genetic materials such as calcium phosphates; and (d) hormones, such as testosterone, estradiol, progesterone, prostaglandins and synthetic analogs thereof; and (e) an anti-cancer drug, such as paclitaxel, doxorubicin, 5-fluorouracil, cisplatin, carboplatin, oxaliplatin, tegafur, irinotecan, docetaxel, cyclophosphamide, gemcitabine, ifosfamide, mitomycin C, vincristine, etoposide, methotrexate, topotecan, tamoxifen, vinorelbine, camptothecin, danuorubicin, chlorambucil, bryostatin-1, calicheamicin, mayatansme, levamisole, DNA recombinant interferon alfa-2a, mitoxantrone, nimustine, interferon alfa-2a, doxifluridine, formestane, leuprolide acetate, megestrol acetate, carmofur, teniposide, bleomycin, carmustine, heptaplatin, exemestane, anastrozole, estramustine, capecitabine, goserelin acetate, polysaccharide potassium, medroxypogesterone acetate, epirubicin, letrozole, pirarubicin, topotecan, altretamine, toremifene citrate, BCNU, taxotere, actinomycin D, polyethylene glycol conjugated protein, and synthetic analogs thereof, and (f) an angiogenesis inhibitor, such as Clodronate, Doxycycline, Marimastat, 2-Methoxyestradiol, Squalamine, Thalidomide, Combretastatin A4, Soy Isoflavone, Enzastaurin, CC 5013 (Revimid; Celgene Corp, Warren, N.J.), Celecoxib, Halofuginone hydrobromide, interferon-alpha, Bevacizumab, Interleukin-12, VEFG-trap, Cetuximab, and synthetic analogs thereof.

Suitable bioactive agents for use in the method of producing a pharmaceutical gel emulsion according to the present invention may further be selected from entities such as (therapeutic) cells, preferably autologous (therapeutic) cells, and may be selected from the group comprising, but not limited to, stem cells, mesenchymal stem cells, precondrocytes, nucleus pulposus cells, preosteoblasts, chondrocytes, umbilical vein endothelial cells (UVEC), osteoblasts, adult stem cells, Schwann cells, oligodendrocytes, hepatocytes, mural cells (used in combination with UVEC), myoblasts, insulin-secreting cells, endothelial cells, smooth muscle cells, fibroblasts, [beta]-cells, endodermal cells, hepatic stem cells, juxraglomerular cells, skeletal muscle cells, keratinocytes, melanocytes, langerhans cells, merkel cells, dermal fibroblasts, and preadipocytes.

In a preferred embodiment of the method of producing a pharmaceutical gel emulsion according to the present invention, the pharmaceutical gel emulsion is foamed.

The pharmaceutical gel emulsion may be foamed by introducing a, preferably pressurized, foaming gas into the pharmaceutical gel emulsion. Suitable foaming propellants include liquefied gases such as hydrocarbons, CFC, hydrochlorofluorocarbons (HCFC), and HFC, where the hydrocarbons include propane, butane, isobutane; other propellants include compressed gases such as nitrogen, nitrous oxide, carbon dioxide, air, inert gases, noble gases, or mixtures thereof. Additionally, a foam stabilizer might be included. Possible foam stabilizers include Sodium Lauryl Sulphate (SLS), Lauric acid, Myristic acid, Palmitic acid, Stearic acid, Coconut oil, Corageenen gum, Stearic monoethonolamine, Gum tragacanth, alginate, Gelatin, sodium CMC, Polyvinyl glycol, Glycerol, Sorbitol Stearic acid, Hydrogenated castor oil, Polysorbate 20,PEG-40 hydrogenated castor oil, Poloxamer F68,Cocamidopropyl betaine, PEG 6 caprylic/capric glycerides, Xanthan gum, Agar, Gaur gum, Hydroxy ethyl cellulose, Hydroxy propyl cellulose, HPMC, Methyl cellulose.

In a preferred embodiment of the method of producing a pharmaceutical gel emulsion according to the present invention, the pharmaceutical gel emulsion is free of a cross-linking agent capable of cross-linking the gelling polysaccharide.

In the case where the gelling polysaccharide is a cellulose ether such as for example carboxymethylcellulose (CMC), the pharmaceutical gel emulsion is free of a cross-linking agent capable of cross-linking the gelling polysaccharide such as di-epoxy or diglycidyl compounds. Exemplary diglycidyl compounds are diglycidyl ethers such as 1,4-butanediol diglycidyl ether or poly(ethylene glycol) diglycidyl ether. An exemplary di-epoxy compound is 1,2,7,8-diepoxyoctane.

In a preferred embodiment of the method of producing a pharmaceutical gel emulsion according to the present invention, the pharmaceutically acceptable oil is comprised in the pharmaceutical gel emulsion in an amount of 2 to 20 weight % based on the total weight of the pharmaceutical gel emulsion, preferably in an amount of 2 to 10 weight % based on the total weight of the pharmaceutical gel emulsion.

In a preferred embodiment of the method of producing a pharmaceutical gel emulsion according to the present invention, the pharmaceutically acceptable oil is a plant oil comprising castor oil or soybean oil and preferably consist of either castor oil or soybean oil.

In a preferred embodiment of the method of producing a pharmaceutical gel emulsion according to the present invention, the emulsifying agent is chosen among phospholipids such as phosphatidyl choline.

In a preferred embodiment of the method of producing a pharmaceutical gel emulsion according to the present invention, the emulsifying agent is comprised in the pharmaceutical gel emulsion in an amount of 0.1 to 2.5 weight % based on the total weight of the pharmaceutical gel emulsion.

In a preferred embodiment of the method of producing a pharmaceutical gel emulsion according to the present invention, the gelling polysaccharide is a plant polysaccharide such as alginate, agarose, or starch, in particular cellulose or a cellulose ether such as carboxymethly cellulose, hydroxypropyl cellulose, or methyl cellulose, or is an animal polysaccharide or derivative thereof such as hyaluronic acid or chitosan.

In a preferred embodiment of the method of producing a pharmaceutical gel emulsion according to the present invention, the gelling polysaccharide is comprised in the pharmaceutical gel emulsion in an amount of 2 to 5 weight % based on the total weight of the pharmaceutical gel emulsion.

In a preferred embodiment of the method of producing a pharmaceutical gel emulsion according to the present invention, the gelling polysaccharide has a molecular weight of about 400 to 800 kDa.

In a preferred embodiment of the method of producing a pharmaceutical gel emulsion according to the present invention, the gelling polysaccharide is a thermally treated polysaccharide such as thermally treated carboxymethly cellulose.

In the context of the present invention, the term “thermally treated polysaccharide” is known to a person skilled in the art; it refers for example to a polysaccharide that has been treated by heating the polysaccharide in vacuo to a temperature between 80° C. and 130° C. for 6 h up to 72 h. In a preferred embodiment, the “thermally treated polysaccharide” is treated at a temperature between 100 and 115° C. for of a duration of 20 up to 30 h.

In a preferred embodiment of the method of producing a pharmaceutical gel emulsion according to the present invention, the polyol is comprised in the pharmaceutical gel emulsion in an amount of 1 to 3 weight % based on the total weight of the pharmaceutical gel emulsion.

In a preferred embodiment of the method of producing a pharmaceutical gel emulsion according to the present invention, the polyol is glycerol.

In a preferred embodiment of the method of producing a pharmaceutical gel emulsion according to the present invention, the aqueous phase is chosen among water, phosphate buffer saline or saline.

In a preferred embodiment of the method of producing a pharmaceutical gel emulsion according to the present invention, the first dispersion and/or the oil-in-water emulsion are formed via microfluidization, turbomixing or sonication.

In a preferred embodiment of the method of producing a pharmaceutical gel emulsion according to the present invention, the bioactive agent is an antibiotic such as gentamycin and/or vancomycin.

It is an object of the present invention to provide a pharmaceutical gel emulsion, preferably obtained by a method according as described above.

It is an object of the present invention to provide a pharmaceutical gel emulsion, preferably obtained by a method as described above, having either 1. a shear storage modulus (G′) above its shear loss modulus (G″), i.e. G′>G″, when measured with a rheometer with a strain sweep at amplitude within the linear viscoelastic range, or 2. a storage modulus above 1000 Pa independently of its loss modulus, when measured with a rheometer with a strain sweep at amplitude within the linear viscoelastic range.

It is an object of the present invention to provide a pharmaceutical gel emulsion, preferably obtained by a method according as described above, having a specific adhesive failure energy of from 0.1 to 1 N/m when measured with an AntonPaar rheometer with P2 geometry executing a wet tack test according to the predefined protocol, wherein the emulsion is an oil-in-water emulsion.

It is an object of the present invention to provide a pharmaceutical gel emulsion, preferably obtained by a method as described above, wherein the emulsion is an oil-in-water emulsion and the pharmaceutical gel emulsion comprises at least an emulsifing agent such as lecithin in an amount of from 1 to 1.5 wt %, an injectable pharmaceutically acceptable oil such as a vegetable or mineral oil in an amount of 8 to 12 wt %, a glycerol in an amount of 1.75 to 2.5 wt % and a, preferably thermally treated, cellulose ether in an amount of from 2.5 to 4.5 wt %, and an aqueous solution.

In a preferred embodiment of the pharmaceutical gel emulsion according to the present invention, the emulsion is an oil-in-water emulsion and the pharmaceutical gel emulsion comprises at least an emulsifying agent such as lecithin in an amount of about 1.2 wt %, a castor oil, soybean oil or a mixture of both an amount of about 10 wt %, glycerol such as oligoethylene glycerol in an amount of about 2.25 wt % and, preferably thermally treated, carboxymethyl cellulose in an amount of from 2.5 to 4 wt %, and an aqueous solution.

In a preferred embodiment of the pharmaceutical gel emulsion according to the present invention, the emulsion is an oil-in-water emulsion and the pharmaceutical gel emulsion comprises at least an emulsifying agent such as lecithin in an amount of 1.2 wt %, a castor oil, soybean oil or a mixture of both an amount of 10 wt %, glycerol such as oligoethylene glycerol in an amount of 2.25 wt % and, preferably thermally treated, carboxymethyl cellulose in an amount of from 2.5 to 4 wt %, and an amount of an aqueous solution or water for injection sufficient to bring the total of amounts to 100 wt %.

It is an object of the present invention to provide a bioactive agent delivery system comprising or consisting of the pharmaceutical gel emulsion, preferably obtainable by the above-mentioned method, which pharmaceutical gel emulsion comprises one or more bioactive agents.

In a preferred embodiment of the pharmaceutical gel emulsion according to the present invention, the pharmaceutical gel emulsion comprises a bioactive agent such an antibiotic, in particular such as gentamicin or vancomycin, in an amount of about 1 and 4 weight percent, respectively, based on the total weight of the pharmaceutical gel emulsion.

It is further an object to provide a pharmaceutical gel emulsion as described above for use in prevention or treatment of conditions including bone fractures, musculoskeletal disorders, orthopaedic conditions, osteosynthesis, and/or joint replacement, resurfacing or preservation. Preferably, such treatment will be applied in the context of internal or external body trauma, in orthopedic surgery, in joint arthroplasty, in a drug delivery system, in an antibiotic delivery system or other an anti-infective delivery systems (including but not limited to additives such as, disinfectants, antimicrobial peptides, quorum sensing inhibitors to prevent biofilm formation, silver (in any pharmaceutical form), nanoparticulates, anti-bacterial adhesins to prevent bacterial attachment, anti MSCRAMMs (microbial surface components recognizing adhesive matrix molecules)) or in the treatment of bone or cartilage defects.

It is further an object to provide a pharmaceutical gel emulsion as described above for use in prevention or treatment of infections, preferably in any of the conditions referred to above. Even more preferably, said pharmaceutical gel emulsion is delivered through extrusion at the site of treatment such as for example at the site of a bone defect, application, surgery and/or other intervention.

It is a further object to provide a pharmaceutical gel emulsion as described above for use during robotic surgery, i.e. with application of the gel emulsion of the present invention by a robotic arm in the context of robotic-assisted surgery with dispensation of the gel emulsion of the present invention for local delivery of active substances for infection prevention, infection treatment, prevention of re-infection, bone healing. Examples of robotic surgeries include but are not limited to robotic joint replacement surgery, joint resurfacing, osteosynthesis, reaming of the intramedullary canal, reaming and aspiration of the intramedullary canal with or without placement of a nail, placement of osteosynthesis devices with contextual dispensation of the gel of the present invention.

It is understood that while the pharmaceutical gel emulsion is stored in an appropriate vessel, it could be molded by hand at the site of the treatment, application, surgery and/or other intervention, after being discharged at, or in the vicinity, of the site of treatment, application, surgery and/or other intervention.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows storage modulus (G′) as a function of strain for composition 1 (line), a grafted hyaluronic acid derivative (cross), CMC (circle), TT-CMC (square).

FIG. 2 shows a typical force-displacement curve, i.e. normal force as a function of gap width, obtained for composition 1 (cross) and a solution of CMC having the same concentration as composition 1 in terms of CMC (circle).

FIG. 3 shows results of the adhesion energy for the tack test of composition 1 in comparison to a solution of CMC having the same concentration as composition 1 in terms of CMC. The reduction in adhesion energy is evident.

FIG. 4 shows the extrusion of a gel emulsion according to the present invention from a syringe into a physiological solution, illustrating the capacity of staying cohesive in water environment.

### DESCRIPTION OF PREFERRED EMBODIMENTS

The gel emulsion according to the present invention may also be suitable for use in the treatment of internal or external body trauma, in orthopedic surgery, in joint arthroplasty, in a drug delivery system, in an antibiotic delivery system or other anti-infective delivery systems (including but not limited to additives such as, disinfectants, antimicrobial peptides, quorum sensing inhibitors to prevent biofilm formation, silver (in any pharmaceutical form), nanoparticulates, anti-bacterial adhesins to prevent bacterial attachment, anti MSCRAMMs (microbial surface components recognizing adhesive matrix molecules)) or in the treatment of bone or cartilage defects.

### EXAMPLES

Samples were prepared to demonstrate the rheological properties of several embodiments of the gel emulsions according to the present invention and the tackiness was determined according to a tack test.

### Example 1

Preparation of Composition 1:

| | Soybean | Soybean | Water for | | | |
|---|---|---|---|---|---|---|
| | Lecithin | Oil | injection | Glycerol | CMC | total |
| Composition 1 | 0.48 g | 4 g | 33.02 g | 0.9 g | 1.6 g | 40 g |
| % | 1.2% | 10.0% | 82.55% | 2.25% | 4.0% | |

Procedure:

The soybean lecithin is dispersed in the water phase and sonicated with an immersion probe ultrasound generator until the dispersion is homogeneous, typically by applying 2 minutes of ultrasound at full power and 50% cycle. The other liquid components (oil and glycerol) are added, and sonication is applied under the same conditions until a homogeneous dispersion is obtained. Carboxymethyl cellulose (CMC) having a molecular weight of 700 kDa is added, and mixed into the dispersion for 3 minutes, allowed to rest for one hour; the cycle of mixing and rest is repeated at least 3 times. The mixture is allowed to rest for 24 h, and finally steam sterilized with a cycle for liquid substances.

### Example 2

Preparation of Composition 2:

| | | Soybean | Water for | | TT- | |
|---|---|---|---|---|---|---|
| Designation | Lecithin | Oil | injection | Glycerol | CMC | total |
| Composition | 0.48 g | 4 g | 33.42 g | 0.9 g | 1.2 g | 40 g |
| 1 | | | | | | |
| % | 1.2% | 10.0% | 83.55% | 2.25% | 3.0% | |

Procedure:

The lecithin is dispersed in the water phase and sonicated with an immersion probe ultrasound generator for 2 minutes at full power and 100% cycle to obtain a homogeneous dispersion. The other liquid components (oil and glycerol) are added, and sonication is applied under the same conditions until a homogeneous dispersion is obtained. The TT-CMC is added, and manually mixed into the dispersion for 3 minutes, allowed to rest for one hour; the cycle of mixing and rest is repeated 3 times. The mixture is allowed to rest for 24 h, and finally steam sterilized with a cycle for liquid substances. Under sterile conditions, the composition is added to Gentamicin Sulfate and Vancomicin to obtain a final concentration of 1% Gentamicin Sulfate and 4% Vancomicin.

The thermally treated CMC (TT-CMC) is obtained by introducing the CMC on a glass vessel, reducing the pressure vacuum and raising the temperature to 110° C. and maintaining said conditions for 24 h to yield thermally treated CMC (TT-CMC).

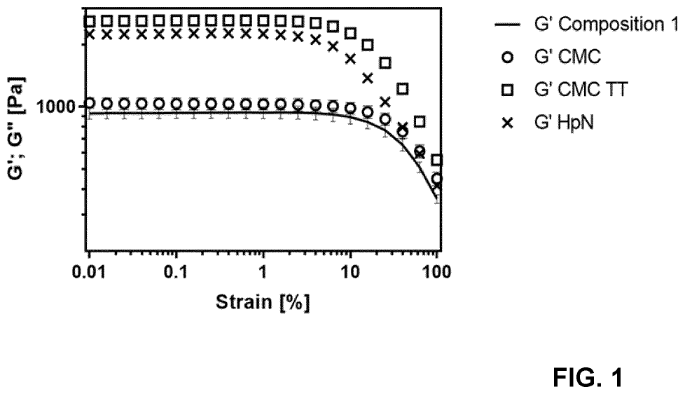

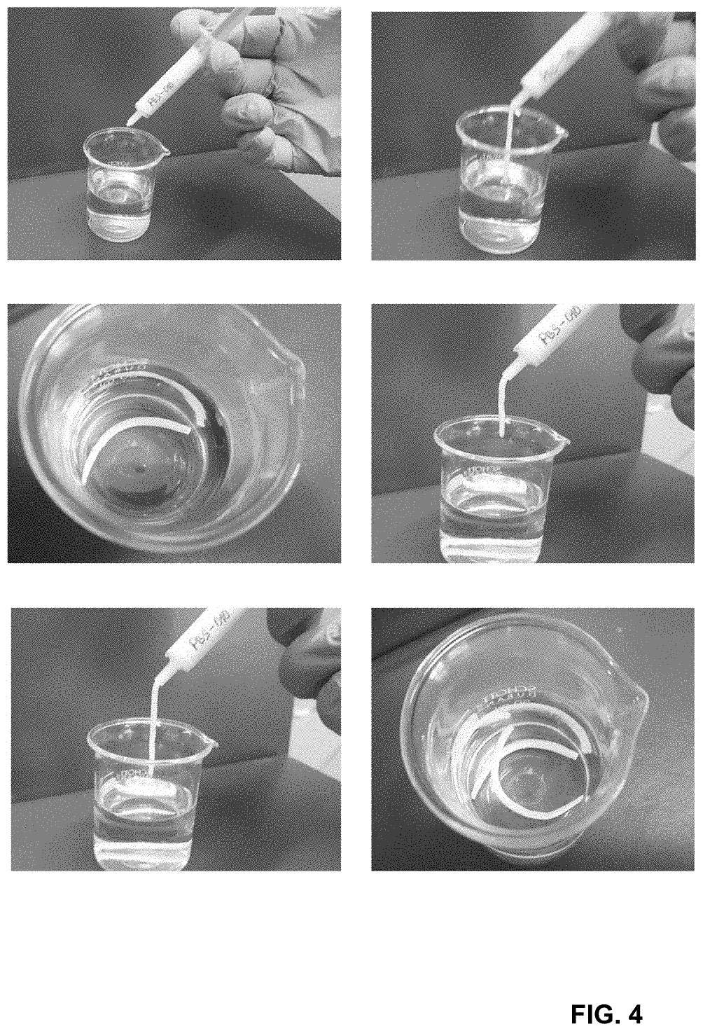

Compound 1 was characterized in terms of rheology, tested for tackiness and compared with other viscoelastic substances used for antibiotic and drug delivery. The measurements are shown in FIGS. 1, 2, 3 and 4.

In FIG. 1, an amplitude sweep test measuring the storage modulus (G′) at 1 rad/sec for a strain varying from 0.01% until 100% was carried out on several samples. As can be seen from FIG. 1, within the linear viscoelastic range, composition 1 (line) has a decrease of G′ of about 7% compared to a standard solution of CMC of the same concentration. A known thermoresponsive hyaluronic acid hydrogel (HpN) obtained as detailed in the paper: M D′Este, M Alini, D Eglin, Carbohydrate polymers 90 (3), 1378-1385 and dissolved 10% w/v was used as a reference in terms of viscoelastic behavior (cross), since it is known to possess a suitable rheological behavior for the application considered in the context of the present invention. Specifically, HpN was used for antibiotic delivery with proven preclinical success. FIG. 1 further shows that while rheological properties of HpN cannot fully be matched by using CMC in 4% in PBS, a match can be achieved using TT-CMC in 4% in PBS.

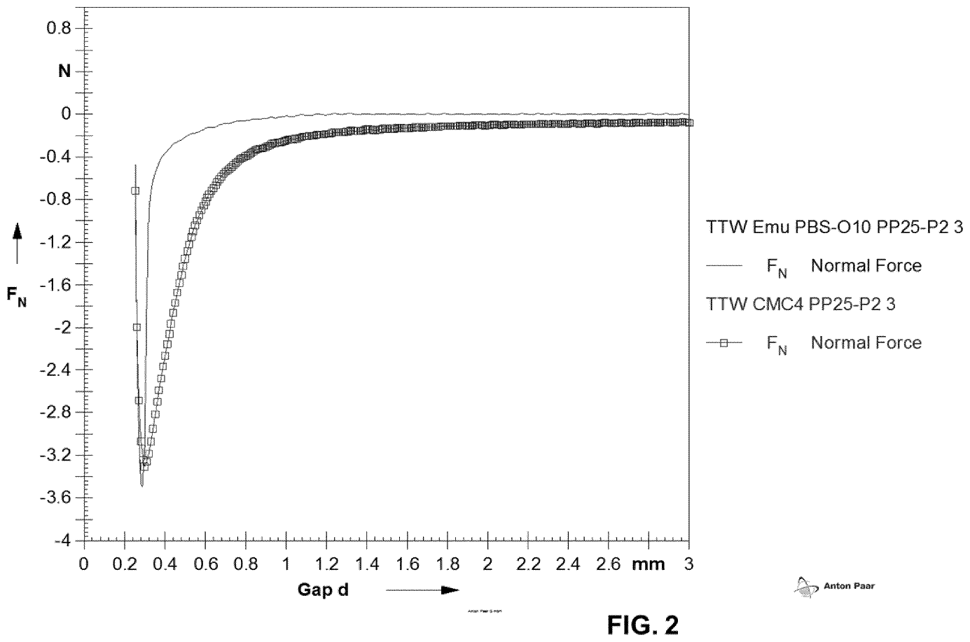

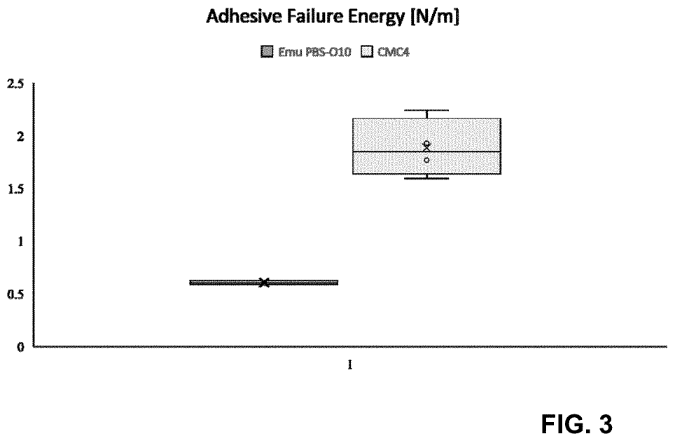

In FIG. 2, the execution of a tack test is reported, where a track of the force as a function of the path is obtained. From the integration of these curves, the adhesive failure energy can be calculated; the adhesion energy so obtained is illustrated in FIG. 3.

In FIG. 3, composition 1 was compared to CMC in a tack test, which measures the adhesive failure energy. The test was performed with an AntonPaar MCR302 rheometer according to the pre-defined protocol, where the substance to be analyzed is inserted in the gap, allowed to equilibrate and finally the plate is pulled up at controlled displacement while measuring the normal force. FIG. 3 shows the results of the tack test for composition 1 in comparison to CMC in 4% PBS. Each substance was tested in triplicate N=3 and the results are shown as box plot. The adhesive failure energy (as calculated by the measurement software by integration of the force/displacement curve) of the CMC in 4% PBS is about 3 times higher than that of the adhesion energy in composition 1. Even though the composition 1 sample comprises the same amount of CMC than the CMC in 4% PBS sample and displays similar viscoelastic properties (see FIG. 1) the adhesion force is significantly lower. This result is very surprising because typically adhesion force is related to viscoelastic properties, i.e. the higher the viscoelastic properties as measured in rheological tests with G′ the higher the adhesion force.

In summary, the compositions of the present invention will stay where placed by the surgeon (owing to the high storage modulus (G′)) and at the same time not stick to the surgeon gloves (owing to the low adhesion force), thereby overcoming a universally recognized limitation of existing antibiotic-loaded biomaterials and/or gels.

### Example 3

Preparation of Composition 3:

| | | Sodium | Soybean | Water for | | | |
|---|---|---|---|---|---|---|---|
| | Lecithin | oleate B | Oil | injection | Glycerol | CMC | total |
| Compo- | 0.48 g | 12 mg | 3 g | 34.01 g | 0.9 g | 1.6 g | 40 g |
| sition 1 | | | | | | | |
| % | 1.2% | 0.003% | 10.0% | 82.55% | 2.25% | 4.0% | |

Egg lecithin with 70-80% phosphatidylcholine is mixed with sodium oleate B in proportion 40:1 and dispersed in the soybean oil until the dispersion is homogeneous, and no clumps are present. Water is combined with glycerol, mixed to homogeneity, combined with the dispersion of phosphatidyl choline and oleate in soybean oil and treated with a high-shear mixer in a temperature range between 25 and 50° C. until a homogeneous and stable dispersion is obtained. The obtained dispersion is steam sterilized for 20 minutes at 121° C. This sterile liquid composition was then delivered to the operating theater, and mixed under sterile conditions with 1.6 g of sterile CMC having a molecular weight of about 500 kDa, daptomycin and gentamicin sulfate to obtain a final concentration of 1% Gentamicin Sulfate and 4% daptomycin. The CMC and the antibiotics are thoroughly mixed until a homogeneous paste is obtained.

### Example 4

Preparation of Composition 4:

| Component | | Soyb | | CMC |
|---|---|---|---|---|
| A | Surfactant | Oil | Gly-water | 0.7 kDa |
| Amount | 1143.75 | 9530 | 63312.5 | 2861.25 |
| (mg) | | | | |

| | Water per | | Gentamicin |
|---|---|---|---|
| Solution B | injection | Vancomycin | sulfate |
| Amount (mg) | 18575 | 3656.25 | 913.75 |

Component A is prepared as follows using the quantities reported in the table above. The surfactant is prepared by mixing egg lecithin of high purity (containing egg phospholipids with 80% phosphatidylcholine) with sodium oleate in ratio 40/1 lecithin/oleate. A solution is prepared dissolving injection-grade glycerol 3.42 weight % in water per injection to obtain the Gly-water solution. The surfactant is dispersed in the soybean oil with a turbo-mixer until homogeneity. The obtained dispersion is combined with the Gly-water solution and mixed under high shear until homogeneity. At this point CMC is incorporated in the dispersion and mixed until homogeneity to obtain Component A, which is steam sterilized and stored at 2-8° C. until use.

Solution B is prepared under sterile conditions by dissolving the indicated amounts of Vancomycin and Gentamicin sulfate in the indicated amount of water per injection.

In order to yield the gel emulsion Composition 4 according to the present invention ready for application at the site of interest, a syringe containing viscoelastic dispersion A is combined with a syringe containing solution B and mixed until homogeneity by means of transferring the solution from one barrel to the other for 30 times to obtain an homogeneous viscoelastic dispersion ready for injection.

### Example 5: Cohesion Test of the Composition 4 Prepared in Example 4

A syringe containing 10 ml of the gel emulsion prepared as per example 4 is prepared. The gel emulsion is extruded through the orifice of the syringe into a water bath at physiological osmolarity at 37° C. As can be seen, the extruded gel emulsion preserves its shape after leaving the syringe, before and after entering a physiological environment, demonstrating the suitability of the gel emulsion of example 4 to be used for injection in the human body and avoiding washing-out from body fluids and displacements from compression by adjacent tissues.

### Example 6: Composition for Intraoperative Mixing with Antibiotics

The same procedure of example 4 is followed, with the only difference of using castor oil instead of soybean oil

### Example 7: Composition for Intraoperative Mixing with Antibiotics

The same procedure of example 4 is followed, with the only difference of employing ultrasound and microfiltration instead of high-shear mixing to obtain the emulsion.

### Example 8: Composition for Intraoperative Mixing with Antibiotics

The same procedure of example 4 is followed, with the only difference of employing purified soy lecithin instead of a combination of egg lecithin and sodium oleate as emulsifier.

### Example 9: Composition for Intraoperative Mixing with Antibiotics

The same procedure of example 4 is followed, with the only difference performing the preparation of the Component A setting the temperature between 40 and 55° C.

### Example 10: Composition for Intraoperative Mixing with Antibiotics

The same procedure of example 4 is followed, with the only difference performing the preparation of the Component A setting the temperature between 5 and 25° C.

### Example 11: Composition for Intraoperative Mixing with Antibiotics

The same procedure of example 4 is followed, with the only difference performing the preparation of the Component A setting the temperature between 25 and 40° C.

### Example 12: Preparation of a Foam for Antibiotics Delivery

The same procedure of example 4 is followed, with the difference that a final concentration of CMC of 1.5% is achieved. The antibiotic-loaded composition is mixed under high-shear incorporating nitrous oxide, and packaged into pressure-tight cylinders to provide a ready-to-use foaming agent.

### Example 13: Composition for Intraoperative Mixing with Antibiotics

The same procedure of example 4 is followed using CMC of molecular weight 700 kDa

### Example 14: Composition for Intraoperative Mixing with Antibiotics

The same procedure of example 4 is followed using CMC of molecular weight 100 kDa.

### Example 15: Composition for Intraoperative Mixing with Antibiotics

The same procedure of example 4 is followed, with the only difference of using purified medical grade olive oil instead of soybean oil.

### Example 16: Composition for Intraoperative Mixing with Antibiotics

The same procedure of example 4 is followed, with the only difference of replacing gentamicin sulfate and vancomycin with rifampicin for a final rifampicin concentration in the final formulation of 4%.

1. A method of producing a pharmaceutical gel emulsion, wherein the emulsion is an oil-in-water gel emulsion, comprising the steps of forming an oil-in-water emulsion comprising at least one pharmaceutically acceptable oil,
at least one aqueous phase, at least one osmotic agent, at least one emulsifying agent, mixing a gelling polysaccharide with the oil-in-water emulsion and allowing the resulting mixture to form the pharmaceutical oil-in-water gel emulsion, optionally mixing an bioactive agent into the oil-in-water pharmaceutical gel emulsion.

2. The method of producing a pharmaceutical gel emulsion according to claim 1, comprising the steps of
a. forming a first dispersion by dispersing at least one emulsifying agent in an aqueous phase,
b. forming an oil-in-water emulsion by dispersing at least one pharmaceutically acceptable oil and at least one osmotic agent in the first dispersion,
c. mixing a gelling polysaccharide with the oil-in-water emulsion and allowing the resulting mixture to form the pharmaceutical gel emulsion,
d. optionally mixing a bioactive agent such as an antibacterial agent into the pharmaceutical gel emulsion.

3. The method of producing a pharmaceutical gel emulsion according to claim 1, wherein the emulsion is an oil-in-water gel emulsion, comprising the steps of
a. forming a first dispersion by dispersing at least one emulsifying agent in at least one pharmaceutically acceptable oil,
b. forming an oil-in-water emulsion by dispersing at least one aqueous phase and at least one osmotic agent in the first dispersion,
c. mixing a gelling polysaccharide with the oil-in-water emulsion and allowing the resulting mixture to form the pharmaceutical gel emulsion,
d. optionally mixing bioactive agent such as an antibacterial agent into the pharmaceutical gel emulsion.

4. The method of producing a pharmaceutical gel emulsion according to claim 1, wherein the pharmaceutical gel emulsion is foamed.

5. The method of producing a pharmaceutical gel emulsion according to claim 1, wherein the pharmaceutical gel emulsion is free of a cross-linking agent capable of cross-linking the gelling polysaccharide.

6. The method of producing a pharmaceutical gel emulsion according to claim 1, wherein the pharmaceutically acceptable oil is comprised in the pharmaceutical gel emulsion in an amount of 2 to 20 weight % based on the total weight of the pharmaceutical gel emulsion.

7. The method of producing a pharmaceutical gel emulsion according to claim 1, wherein the pharmaceutically acceptable oil is a plant oil comprising castor oil or soybean oil.

8. The method of producing a pharmaceutical gel emulsion according to claim 1, wherein the emulsifying agent is chosen among phospholipids such as phosphatidyl choline.

9. The method of producing a pharmaceutical gel emulsion according to claim 1, wherein the emulsifying agent is comprised in the pharmaceutical gel emulsion in an amount of 0.1 to 2.5 weight % based on the total weight of the pharmaceutical gel emulsion.

10. The method of producing a pharmaceutical gel emulsion according to claim 1, wherein the gelling polysaccharide is alginate, agarose, or starch; or carboxymethly cellulose, hydroxypropyl cellulose, or methyl cellulose; or hyaluronic acid, or chitosan.

11. The method of producing a pharmaceutical gel emulsion according to claim 1, wherein the gelling polysaccharide is comprised in the pharmaceutical gel emulsion in an amount of 2 to 5 weight % based on the total weight of the pharmaceutical gel emulsion and/or has a molecular weight of about 400 to 800 kDa.

12. The method of producing a pharmaceutical gel emulsion according to claim 1, wherein the gelling polysaccharide is thermally treated carboxymethly cellulose.

13. The method of producing a pharmaceutical gel emulsion according to claim 1, wherein the osmotic agent is a polyol, and is comprised in the pharmaceutical gel emulsion in an amount of 1 to 3 weight % based on the total weight of the pharmaceutical gel emulsion.

14. The method of producing a pharmaceutical gel emulsion according to claim 1, wherein the osmotic agent is a polyol, preferably a glycerol such as polyethylene glycerol or polypropylene glycerol.

15. The method of producing a pharmaceutical gel emulsion according to claim 1, wherein the at least one aqueous phase is chosen among water, phosphate buffered saline or saline.

16. The method of producing a pharmaceutical gel emulsion according to claim 1, wherein the first dispersion and/or the oil-in-water emulsion are formed via microfluidization, microfiltration or sonication.

17. The method of producing a pharmaceutical gel emulsion according to claim 1, wherein the bioactive agent is an antibiotic such as gentamycin and/or vancomycin.

18. A pharmaceutical gel emulsion obtained by a method according to claim 1.

19. A pharmaceutical gel emulsion, obtained by a method according to claim 1, having a shear storage modulus (G′) above its shear loss modulus (G″), i.e. G′>G″, when measured with a rheometer with a strain sweep at amplitude within the linear viscoelastic range, wherein the emulsion is an oil-in-water emulsion.

20. A pharmaceutical gel emulsion, obtained by a method according to claim 1, having an adhesive failure energy of from 0.1 to 1 N/m, when measured with an AntonPaar rheometer with P2 geometry executing a wet tack test, wherein the emulsion is an oil-in-water emulsion, wherein the emulsion is an oil-in-water emulsion.

21. A pharmaceutical gel emulsion, obtained by a method according to claim 1, wherein the emulsion is an oil-in-water emulsion and the pharmaceutical gel emulsion comprises at least lecithin in an amount of from 1 to 1.5 wt %, a vegetable oil in an amount of 8 to 12 wt %, a glycerol in an amount of 1.75 to 2.5 wt % and a cellulose ether in an amount of from 2.5 to 4.5 wt %, and an aqueous solution.

22. The pharmaceutical gel emulsion, obtained by a method according to claim 1, wherein the emulsion is an oil-in-water emulsion and the pharmaceutical gel emulsion comprises at least lecithin in an amount of about 1 wt %, a castor oil, soybean oil or a mixture of both an amount of about 10 wt %, a polyethylene glycol in an amount of about 2.25 wt % and carboxymethyl cellulose in an amount of from 3 to 4 wt %, and an aqueous solution.

23. The pharmaceutical gel emulsion, obtained by a method according to claim 1, wherein the emulsion is an oil-in-water emulsion and the pharmaceutical gel emulsion comprises at least lecithin in an amount of 1 wt %, a castor oil, soybean oil or a mixture of both an amount of 10 wt %, a polyethylene glycol in an amount of 2.25 wt % and carboxymethyl cellulose in an amount of from 3 to 4 wt %, and phosphate buffer saline in an amount sufficient to bring the total of amounts to 100 wt %.

24. A method of treatment of an infection in the context of a bone fracture, orthopaedic condition, osteosynthesis, and/or joint replacement or preservation, comprising the step of administering a pharmaceutical gel emulsion, obtained by a method according to claim 1.

25. A method of prevention of an infection in the context of a bone fracture, orthopaedic condition, osteosynthesis, and/or joint replacement or preservation, comprising the step of administering a pharmaceutical gel emulsion, obtained by a method according to claim 1.

26. The method of producing a pharmaceutical gel emulsion according to claim 1, wherein the pharmaceutically acceptable oil is comprised in the pharmaceutical gel emulsion in an amount of 2 to 10 weight % based on the total weight of the pharmaceutical gel emulsion.
